(19) United States
(12) Patent Application Publication
(10) Pub. No.: US 2024/0008860 A1
(43) Pub. Date: Jan. 11, 2024

(54) Surgical Tissue Orientation Clip, Delivery Device, System and Method for Using the Same

(71) Applicant: Vector Surgical, LLC, Waukesha, WI (US)

(72) Inventors: Janet L. F. Phillips, Nashotah, WI (US); Glenn Walters, Port Washington, WI (US)

(21) Appl. No.: 18/042,012

(22) PCT Filed: Aug. 24, 2021
(86) PCT No.: PCT/US21/47267

§ 371 Date: Feb. 17, 2023

Related U.S. Application Data
(60) Provisional application No. 63/069,357, filed on Aug. 24, 2020.

(51) Int. Cl. A61B 10/02

(52) U.S. Cl. CPC A61B 10/0266; A61B 2010/0208; A61B 2010/0225

(57) ABSTRACT

A tissue orientation clip and a delivery device that delivers one or more tissue orientation clips to a tissue site are provided. The tissue orientation clip includes a first clip jaw and a second clip jaw, with each including one or more teeth, and includes a first lateral handle positioned on a proximal end of the first clip jaw, and a second lateral handle positioned on a proximal end of the first clip jaw. A method of delivering one or more tissue orientation clips to a tissue specimen is also disclosed.

### CROSS-REFERENCE TO RELATED APPLICATION

This application claims priority benefit under 35 U.S.C. § 119(e) to U.S. Provisional Application Ser. No. 63/069,357, filed Aug. 24, 2020, the contents of which is incorporated by reference herein in its entirety.

### FIELD OF THE DISCLOSURE

A tissue orientation clip, applicator and system including the clip and applicator are disclosed. The clips may be inserted using the applicator disclosed herein and used in minimally invasive surgery and/or with small excised tissue specimens, where the clips indicate the original positioning of the tissue in the patient's body. This orientation information of the tissue can be extremely important to both radiographic and pathology analysis of the tissue. The applicator may be used to deliver the clip to the tissue to be excised either immediately before, during, or after excision.

### BACKGROUND OF THE DISCLOSURE

The goal of tissue conserving cancer surgery is to remove the cancerous tissue while retaining as much healthy tissue as possible. The excised tissue is carefully examined (e.g., radiographic or pathology analysis) to verify that a sufficient volume of healthy tissue surrounding the tumor was removed. To determine this, the tissue sample is examined and the distance between the cancerous cells and the exterior surface of the specimen are identified. If the amount of healthy tissue between the cancerous cells and the exterior surface of the specimen is insufficient, or too small, then the analysis is considered “positive,” and a second surgery is often performed to remove additional tissue. In this case, it is imperative that the surgeon know the original orientation of the tissue as it was positioned in the patient's body so that he or she can remove additional tissue in the correct location to successfully excise all of the cancer.

Tissue specimens are analyzed in terms of “margins,” which are portions of the exterior surface of the excised specimen. A spherical tissue specimen is typically described by reference to six margins (anterior, posterior, lateral, medial, superior and inferior). Some anatomical regions of the body require different labeling of the specimen margins, such as radial versus ulnar or proximal versus distal. In any case, precise indication of the margin is essential when radiology or pathology analysis indicates that there is insufficient volume of healthy tissue between the cancer and the outer surface of the specimen, as it pinpoints the location where cancer may remain in the body.

Precise tissue orientation, or the labeling of excised tissue margins to indicate how the specimen was originally located within the body, is critical to accurate feedback from radiologic and pathology analysis of the excise tissue. Orientation of the tissue allows the radiology or pathology report to specify which “margin,” or surface area of the excised tissue, featured the positive edge close to the cancer. A positive report is often treated with a subsequent action to remove more tissue; in other cases it may direct subsequent treatment such as post-surgical radiation therapy. Surgical re-excision may occur during a surgical case, as when a specimen radiograph or a “frozen section” is performed to analyze the tissue margins, or in a subsequent surgery, as with traditional pathology analysis of microscopic slides of the tissue.

In an open surgical case, such as a breast lumpectomy, the surgeon can easily view the tissue as he or she is excising it from the patient's body. In these cases, the surgeon removes the tissue and then readily applies a marking ink, such as that disclosed in U.S. Patent Publication No. 2017/0189135, incorporated herein by reference in its entirety herein, optionally in conjunction with tissue orientation clips to define the specimen margins that indicate original orientation of the tissue as it was in the patient. In open surgical cases, the size of the excised specimen can affect the properties of how specimens are most effectively marked for orientation. While the clips disclosed in U.S. Pat. No. 8,594,768, are well suited to specimens larger than 20 cm³, they are too large for use with specimens <20 cm³ and designed to be used after the tissue is excised from the body.

In minimally invasive surgery, including endoscopic, laparoscopic and robotic cases, information on the original orientation of the tissue prior to its excision is often lost. The surgeon is not able to visually track the positioning of the tissue as it is excised and removed through a trocar or other equipment. During the process of tissue removal, several forces can act upon the specimen that compromise, distort, or result in the loss of orientation information. First, the shape of the specimen may be altered due to stretching or compression as it is pulled through the trocar, which may range in size from about 8 mm to about 12 mm. Second, the tissue may be twisted or rotated as it is extracted, also contributing to a loss of understanding regarding how the specimen was originally shaped and where it was originally positioned in the body. Further, in some cases, the specimen may be morcellated, or cut into small pieces, prior to removal; this also contributes to a loss of information on the original specimen margins. Finally, due to the challenge of manipulating the tissue using the equipment in these cases, the tissue may be dropped within the surgical cavity prior to removal, which also results in a loss of orientation. Orientation information may also be lost after the specimen is removed. Tissue “relaxes” its shape when it is no longer surrounded by the supporting architecture of adjacent tissue. Further, if a specimen is submerged in formalin prior to analysis, it can harden and contract, changing the size and shape of the specimen, and disturbing the margin surfaces.

Small vessel clips, e.g. hemoclips, may be used to indicate margin orientation. However, these clips are used throughout surgery, and may be easily confused with the same type of clip used for blood vessels; they have no inherently unique or differentiating feature which might indicate a margin or point of interest and may not securely attach to tissue. Further, or may require that the specimen be cut or altered in order for removal for pathology analysis, which can distort the margin planes.

A tissue orientation clip system is needed that can be used in both minimally invasive procedures and open surgeries to address the problems noted above and that can be used with tissue to identify orientation prior to and after excising it from the body.

### SUMMARY OF THE DISCLOSURE

A tissue orientation clip is disclosed herein including: a first clip jaw including one or more teeth at a distal end of the first clip jaw, a first lateral handle at a proximal end of the first clip jaw, the first lateral handle defining a first spring arm receiving slot therein; a second clip jaw including one or more teeth at a distal end of the second clip jaw, a second lateral handle at a proximal end of the second clip jaw, the second lateral handle having a second spring arm receiving slot therein; and a spring including a first spring arm received in the first spring arm receiving slot and a second spring arm received within the second spring arm receiving slot. The spring may be a torsion spring or a U-shaped spring that biases the tissue orientation clip in a closed position. The spring may be a torsion spring including a circular spring portion defining a spring aperture therethrough, the circular spring portion being at least partially received within the first clip jaw and the second clip jaw. There may be a protrusion in the second clip jaw which may be rotatably received in a protrusion receiving hole in the first clip jaw. The tissue orientation clip may include a retaining clip received by the first clip jaw and the second clip jaw. The retaining clip may include a removal tab for removing the retaining clip from the clip jaws prior to use. The first clip jaw may include one or two teeth and the second clip jaw may include one or two teeth. The first clip jaw may include two teeth and the second clip jaw may include one tooth.

The clip may include indicia, optionally radiopaque, indicating an orientation of a tissue specimen. A color of the tissue orientation clip may be selected from red, orange, yellow, blue, green, violet, black, gold, silver teal, magenta, white, light blue, pink, periwinkle and marigold, optionally wherein each color represents a margin of a tissue specimen selected from any known margin, such as, but not limited to, anterior, posterior, medial, lateral, superior, inferior, radial, ulnar, proximal and distal.

The tissue orientation clip is sized to be smaller in both the closed and open positions than an inner diameter of a trocar through which the tissue orientation clip will be extracted.

Tissue orientation clip delivery systems for delivering one or multiple tissue orientation clips are disclosed. A delivery system for delivering a tissue orientation clip, comprises: a delivery device including: an outer sheath; a plunger disposed in the outer sheath; and a clip shield coupled to a distal end of the outer sheath; and a tissue orientation clip. The tissue orientation clip is positioned adjacent a distal end of the plunger, and the tissue orientation clip is coupled within the clip shield. A proximal end of the outer sheath may include a finger-graspable handle. A proximal end of the plunger may include a clip release button. The delivery device may further include a spring surrounding an outer circumference of the plunger and disposed between the clip release button and the outer sheath and/or a button lock including a removal tag configured to prevent clip deployment prior to use. The first and second lateral handles of the tissue orientation clip may be coupled within the clip shield. A second clip jaw of the tissue orientation clip may include at least one post extending therefrom, that is received by a track within the clip shield.

A delivery system housing multiple tissue orientation clips may comprise: a delivery device including: a cartridge including a distal end and a proximal end, and a cartridge lumen therewithin; a plunger including a plurality of teeth; and a trigger handle including an extension arm and a catch engageable with a tooth of the plurality of teeth; and a plurality of tissue orientation clips housed within the cartridge lumen at a distal end thereof. A number of teeth in the plurality of teeth on the plunger may correspond to a number of the plurality of clips in the cartridge lumen. The plurality of tissue orientation clips may be biased in the open position in the cartridge lumen or biased in the closed position in the cartridge lumen.

The cartridge may include an aperture for receiving a removable pin configured to prevent plunger from moving prior to use. The cartridge lumen may include a track along the length of the cartridge, or two tracks along the length of each of the opposing sides of the cartridge. Each of the plurality of tissue orientation clips may include a post extending therefrom, an end of each post is received by the track. The post may extend from a first lateral handle, a second lateral handle, a first clip jaw and/or a second clip jaw. The second clip jaw may include at least one post extending from said second clip jaw, and wherein the at least one post is received by the track. Alternatively, the first lateral handle and the second lateral handle may include a first elongate extension post and a second elongate extension post respectively coupled thereto. When the cartridge lumen includes upper tracks along the length of each of the opposing sides of the cartridge and lower tracks along the length of each of the opposing sides of the cartridge, the upper tracks and lower tracks may receive the first and second elongate extension posts.

The trigger handle extension post may include a trigger handle protrusion matingly receivable by an aperture in the cartridge. The trigger handle may include a trigger handle body having a trigger lever pivotally coupled thereto, the trigger lever biased in a non-actuated position by a compression spring housed within the trigger handle body, and/or a stop lever configured to engage ratcheting slots on the plunger to prevent plunger from retracting proximally after being actuated by a trigger pull.

The plunger may include a first plunger positioned in the cartridge lumen having a distal end contacting the most proximally positioned tissue orientation clip, and a second plunger including a distal end that when assembled to the trigger handle is in contact with the proximal end of the first plunger, and/or numerical indicia on the plunger to indicate the number of tissue orientation clips remaining in the delivery device. A proximal end of the plunger may extend past the proximal end of the cartridge and be received within an upper channel of the trigger handle.

Also disclosed is a method of delivering one or more tissue orientation clips to a target site using a delivery system including a delivery device and at least one tissue orientation clip. The delivery device may include: a cartridge including a distal end and a proximal end, and a cartridge lumen therewithin; a plunger including a plurality of teeth; and a trigger handle including an extension arm and a catch engageable with a tooth of the plurality of teeth. The method includes providing the delivery system that may include the delivery device and a plurality of tissue orientation clips housed within the cartridge lumen at a distal end thereof; inserting the delivery device proximate to the target site; and expelling a first tissue orientation clip of the plurality of tissue orientation clips from the distal end of the cartridge by actuating the trigger handle to move the catch and advance the plunger. Expelling the first clip may include squeezing a trigger lever on the trigger handle and/or sliding a post extending from the first clip along a track defined along the cartridge and/or transitioning the first clip from an open position to a closed position on the tissue site. When the first tissue orientation clip is expelled, a second tissue orientation clip may advance to the distal end of the cartridge and/or a number displayed in a window defined by the trigger handle changes. By advancing the plunger, the first tissue orientation clip is pushed out of the distal end of the cartridge.

The method may include providing the delivery system that includes a single-clip delivery device and one tissue orientation clip positioned adjacent a distal end of the delivery device, and coupled to the delivery device. The single-clip delivery device may include: an outer sheath, a finger-graspable handle engaged to the outer sheath, a plunger having a proximal end and a distal end, the plunger disposed on the outer sheath, and a clip release button engaged to the proximal end of the plunger. This method may include: providing the delivery system; inserting the delivery system in proximity to the target site; actuating the clip release button on the delivery device to move the plunger in the distal direction; and expelling the tissue orientation clip from a distal end of the plunger.

### BRIEF DESCRIPTION OF THE DRAWINGS

FIG. 1 is a view of a patient for whom a specimen of tissue will be marked and removed.

FIG. 2 is a drawing of a tissue specimen with different surfaces being denoted but not yet marked with one or more clips.

FIG. 3 is the tissue specimen of FIG. 2 that is not marked with three tissue orientation clips.

FIG. 4 is a perspective view of a tissue orientation clip in an open position.

FIGS. 5-6 are exploded views of the tissue orientation clip of FIG. 4.

FIG. 7 is a perspective view of a second embodiment of a tissue orientation clip with a U-shaped spring in an open position.

FIG. 8 is a perspective view of a third embodiment of a tissue orientation clip having first and second extension posts in an open position.

FIG. 9 is a side view of the tissue orientation clip of FIG. 8.

FIG. 10 is a perspective view of a fourth embodiment of a tissue orientation clip having a cuboid-shaped first lateral handle in an open position.

FIG. 11 is a perspective view of the tissue orientation clip of FIG. 10 in a closed position.

FIG. 12 is a rear-ward perspective view of the tissue orientation clip of FIG. 10.

FIG. 13 is a perspective view of a first clip jaw of the tissue orientation clip of FIG. 10.

FIG. 14 is a perspective view of a second clip jaw of the tissue orientation clip of FIG. 10.

FIG. 15 is a side view of a delivery device for a tissue orientation clip.

FIG. 16 is a perspective view of a distal end of the delivery device of FIG. 15.

FIG. 17 is a cross-sectional view of the distal end of the delivery device of FIG. 16.

FIG. 18A is a cross-sectional view of a distal end of a second embodiment of a delivery device housing the tissue orientation clip of FIG. 10.

FIG. 18B is a cross-sectional view of the delivery device of FIG. 18A.

FIG. 19 is a cross-sectional view of a distal end of a third embodiment of a delivery device housing the tissue orientation clip of FIG. 7.

FIG. 20 is a perspective view of a delivery device configured for housing multiple tissue orientation clips.

FIG. 21 is a proximal perspective view of the delivery device of FIG. 20.

FIG. 22 is atop perspective view of the delivery device of FIG. 20.

FIG. 23 is a cross-sectional view of the delivery device of FIG. 20.

FIG. 24 is a perspective view of a distal end of the delivery device of FIG. 20 housing multiple tissue orientation clips.

FIG. 25 is a front view of the distal end of the delivery device of FIG. 24.

FIG. 26 is a side view of a distal end of a second embodiment of a delivery device housing multiple tissue orientation clips.

FIG. 27 is a perspective view of a third embodiment of a delivery device housing multiple tissue orientation clips of FIG. 10.

FIG. 28 is a perspective view of a distal end of the delivery device of FIG. 27 with a tissue orientation clip being expelled therefrom.

FIGS. 29-30 are perspective views of a distal end of the delivery device of FIG. 19 with the tissue orientation clip being expelled therefrom.

### DETAILED DESCRIPTION

The tissue orientation clip system in accordance with the disclosure comprises one or more tissue orientation clips that are applied to tissue within the surgical cavity just prior to or during excision. A clip applicator or delivery device for attaching a single clip or multiple clips to a tissue specimen is also described. The clips mark one or more margins of the specimen prior to complete excision, thereby preventing orientation information from being lost. The clips grasp the tissue quickly and securely, while resulting in minimal damage to the tissue. Due to the automatic action of the delivery device, the clips can be applied to tissue without the need for an additional tool to support the tissue that is being clipped; in minimally invasive surgery, parsimony of equipment is helpful since there is limited access to the surgical cavity. The clips reside inside the patient for only a short time during the surgical procedure (typically less than ten minutes) while the tissue is being excised. The clips may be color coded and may be packaged in a set of from three to eight or more different colors, where each color represents a different specimen margin, or they may be marked with an indicia to represent the margin, such that a unique indicia represents each specimen margin. For example, each different color or indicia represents a margin of a tissue specimen selected from anterior, posterior, medial, lateral, superior, inferior, radial, ulnar, proximal, distal, superficial, deep, cranial, caudal, canthal, dorsal, planter, ventral, endometrial, cervical, apex, base, right, left, retroperitoneal, costal, helical, palmer, radial, toward lesion, scalp, ileal, esophageal, nipple, subareolar, chest wall, axillary, midline, isthmus, bronchial, ureteral, bladder base, urethral, vas deferens, pancreatic duct, cystic duct, duodenal, gastric, esophageal, ileal, ampullary, pyloric, vascular groove, or all clock faces (esp. 3, 6, 9, 12 o'clock). Each different color or indicia may represent a tissue specimen of the pancreas (superior mesenteric vein, pancreatic stump, biliary duct, and superior mesenteric artery), sarcoma (ulnar and radial), or other area of interest (e.g., nipple-areola complex, close to nerve, and close to bone). Representative colors include red, orange, yellow, blue, green, violet, black, gold, silver teal, magenta, white, light blue, pink, periwinkle, marigold and other colors known to those of skill in the art. The clip may be colorless and translucent. The clips can easily be removed manually after the tissue is fully excised. Each different color or indicia may be used to communicate clinical information, such as a tissue margin or an area of interest for further analysis.

The clip delivery system disclosed herein allows for delivery of clips to small specimens and is useable through equipment such as a trocar. The clips attach securely and are easily removed from the surgical site, using minimally invasive trocars or other instruments, while staying attached to the tissue. Indicia or color features of the clips allow identification of particular tissue margins or anatomical features. An applicator, also referred to herein as a “delivery device,” that is able to deliver a single clip or multiple clips without the need to “reload” the applicator after expelling each clip is also disclosed.

Tissue specimens that are approximately <20 cm³ are best marked using small clips that are less than about 3.5 cm in length by less than about 3.5 cm in width, as disclosed herein. These small tissue orientation clips can attach to the smaller specimen without the clip prongs piercing an adjacent margin and without undue damage to the specimen. Handling of the specimen that is approximately <20 cm³ and interpretation of the specimen margins for correct orientation is also facilitated using a small clip, i.e., the tissue orientation clips disclosed herein. In addition, unlike a larger device, the small clip is compatible with application to tissue and extraction through a trocar or other minimally invasive equipment.

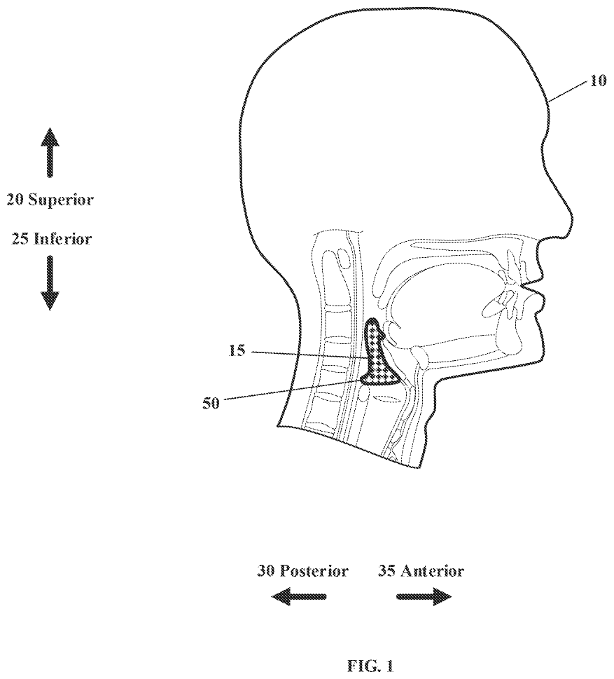

With reference to FIGS. 1-3, a patient 10 and a tissue specimen 15 are illustrated to show the tissue specimen's orientation in the patient 10. Before proceeding, it is contemplated that the tissue orientation clip in accordance with the disclosure is designed to mark a tissue specimen 15 in situ before it is removed, for example, from an area of the neck. However, one of ordinary skill in the art will appreciate that the tissue orientation clip is designed to be manually applied to the tissue specimen after it has been removed from the body. Further, those of skill in the art will appreciate that the tissue orientation clip in accordance with the disclosure may also be applicable to any tissue specimen, other than from the neck region, in which marking for future clinical attention or tissue orientation is desired.

For example, pancreatic cancer surgery also requires that a tissue specimen be removed, and that its orientation be identified to precisely analyze the margins. As such, the invention should not be limited only to the uses described herein as it is well suited for use with any tissue that required orientation for pathology and/or radiology. These tissues include but are not limited to specimens of head and neck, pancreas, thyroid, lymph nodes, brain, sarcomas, kidney, bowel, spleen, soft tissue masses, melanoma, squamous cell skin cancer, basal cell cancer, liver tumors, breast, and the like.

FIG. 1 illustrates a tissue specimen 15 within the patient 10 (side view) prior to its removal and prior to delivery of a tissue orientation clip of the disclosure. For purposes of description, the direction 20 toward the top of the patient's head will be identified as superior, while the opposite direction 25 is inferior. The direction toward the back of the head 30 is defined as posterior, while the direction toward the front of the head or face 35 is defined as anterior.

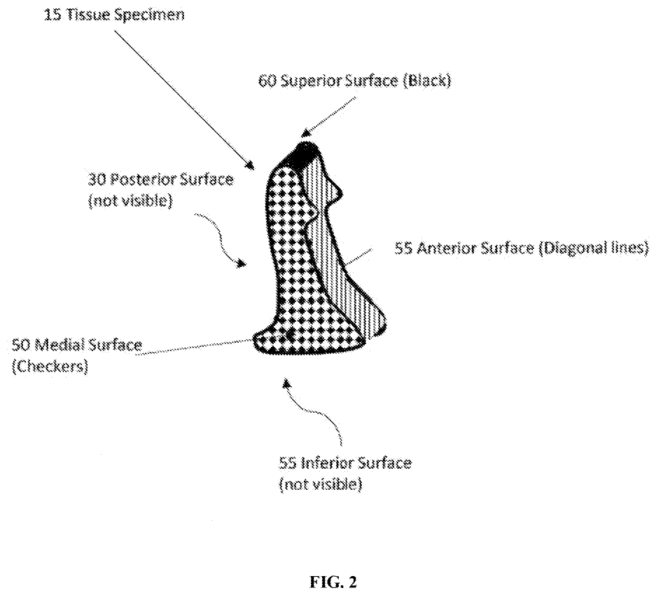

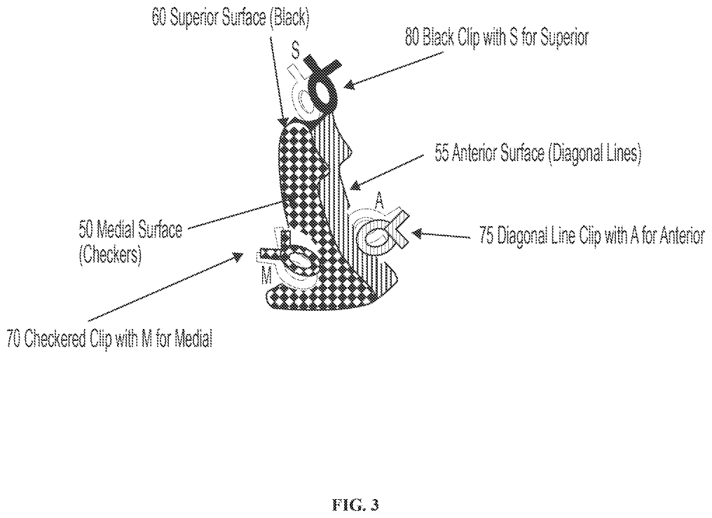

FIG. 2 shows that same tissue specimen 15 still in the patient 10. The surface 50 that will be marked can be seen. For spherically shaped specimens, a minimum of three orthogonal surfaces of tissue specimen 15 can be used to identify the orientation of the specimen 15, however some applications with spherical specimens may involve marking six surfaces. In FIGS. 2 and 3, the medial surface 50, anterior surface 55, and superior surface 60 of the tissue specimen 15 are shaded differently for illustrative purposes. In FIG. 3, clips 70, 75, 80 are attached to surfaces 50, 55, 60 to indicate tissue orientation. For specimen shapes of greater complexity, more than three surfaces, for example, four, five or six surfaces, may be marked.

An embodiment of the disclosure is a tissue orientation clip system. The system includes clips and a delivery device or applicator both of which may be sterile and single-use. In other embodiments, the clips may be single-use while the applicator may have multiple uses. In still other embodiments, both the clips and the applicator may be capable of being re-sterilized and have multiple uses. The clips may be constructed of a strong, light, material that is easily colored such as a gamma stable plastic, such as HPSI-1124, titanium, or stainless steel that is anodized to achieve the desired colors. The clips may be constructed of a gamma stable plastic selected from an aromatic polyester (e.g., PET, PETG), polystyrene (PS), polymethylmethacrylate (PMMA), acrylonitrile butadiene styrene (ABS), polycarbonate (PC), or any combination thereof.

The applicator may also be constructed of gamma stable plastic, such as an aromatic polyester (e.g., PET, PETG), polystyrene (PS), polymethylmethacrylate (PMMA), acrylonitrile butadiene styrene (ABS), polycarbonate (PC), or any combination thereof, that allows for lubricity between the interior plunger and the lumen of the exterior sheath as more fully described below. The applicator is designed to fit through medium to large trocars, which are about 8 mm-about 12 mm in diameter, for delivery to the tissue site. The clips may alternatively be smaller in size and used with other equipment having a smaller diameter. The tissue orientation clips are sized to fit within the cartridge part of the applicator or in other words are smaller than the inner diameter of the applicator lumen. The tissue orientation clips are also sized to be smaller than the inner diameter of the trocar through which the tissue specimen will be extracted. The tissue orientation clips may be less than about 35 mm in length and less than about 35 mm in width. The tissue orientation clips may be about 1 mm to about 30 mm, about 2 mm to about 20 mm, about 3 mm to about 15 mm, or about 4 mm to about 9 mm in width. The tissue orientation clips may be about 5 mm to about 30 mm, about 8 mm to about 20 mm, or about 9 mm to about 16 mm in length. A width of the first clip jaw (including or not including any posts extending therefrom) may be the same or different from a width of the first lateral handle. In an embodiment, a width of the first clip jaw not including any posts extending therefrom may be narrower than the width of the first lateral handle. The length of the first lateral handle may be the same or different from the length of the second lateral handle. In an embodiment, a length of the first lateral handle may be longer than the length of the second lateral handle.

The clips may be different colors, each color representing one margin of a tissue specimen. Each tissue orientation clip may be made of any desirable color, such as red, orange, yellow, blue, green, violet, black, gold, silver teal, magenta, white, light blue, pink, periwinkle, marigold, or be colorless/translucent. Each tissue orientation clip may be made of multiple colors. Each color may be selected to represent a designated margin of a tissue specimen, including but not limited to: proximal, distal, superficial, deep, cranial, caudal, canthal, dorsal, planter, ventral, endometrial, cervical, apex, base, right, left, retroperitoneal, costal, helical, palmer, radial, toward lesion, scalp, ileal, esophageal, and all clock faces, or tissue of the pancreas, sarcoma or other area of interest. However, those of skill in the art will appreciate that the variations of color that may be used are limitless and fall within the scope of the invention. In some cases, the clips are used with a marking ink that marks the tissue specimen such as those described in U.S. Pat. Nos. 10,238,465 and 10,507,777 (the entireties of which are hereby incorporated by reference) and the color of each clip matches the color of a marking ink to represent a particular margin plane. For example, a red clip and a red ink may both represent the superior margin. The red clip is applied in the surgical cavity before the tissue is fully excised to maintain the information. Once the tissue is excised, red ink is applied to the tissue on the margin where the red clip is located. Alternatively, both the red ink and the red clip may be applied after excision of the specimen. In this case, the red ink would mark the tissue for pathology analysis, and the red clip would feature a radio-opaque indicia.

A clip may be applied to tissue immediately prior to removal of the tissue from the patient. This preserves the information on the shape, direction, and location of the tissue before it risks being lost during excision due to the factors cited above. Several clips may be applied to a specimen before being removed from the patient; this is particularly likely if the specimen is morcellated, or cut into smaller pieces, before removal from the surgical cavity. Then the surgeon can use inks to articulate the complete shape and size of each specimen, indicating how the various pieces of tissue removed relate to one another, and the margin planes for each before the tissue is sent to pathology. Alternatively, the surgeon may rely solely on the clips to designate the margin planes of each specimen and how various pieces of excised tissue relate to one another.

An additional, differentiated clip color, by way of non-limiting example gold or silver, may indicate an “area of interest.” This is an area of tissue that the surgeon marks to ensure that it receives attention during pathology or radiology analysis. For example, it may be near a vessel or nerve located close to where cancer was present, but where the surgeon cannot remove tissue without damaging critical anatomical structures.

An embodiment is a tissue orientation clip including: a first clip jaw including one or more teeth at a distal end of the first clip jaw, a first lateral handle at a proximal end of the first clip jaw, the first lateral handle defining a first spring arm receiving slot therein; a second clip jaw including one or more teeth at a distal end of the second clip jaw, a second lateral handle at a proximal end of the second clip jaw, the second lateral handle having a second spring arm receiving slot therein; and a spring including a first spring arm received in the first spring arm receiving slot and a second spring arm received within the second spring arm receiving slot. The spring may be a torsion spring or a U-shaped spring that biases the tissue orientation clip in a closed position. The spring may be a torsion spring including a circular spring portion defining a spring aperture therethrough, the circular spring portion being at least partially received within the first clip jaw and the second clip jaw. Alternatively, the spring may be coupled to or attached to the first clip jaw and the second clip jaw.

There may be a protrusion in the second clip jaw which may be rotatably received in a protrusion receiving hole in the first clip jaw.

The first clip jaw and the second clip jaw may have the same number of teeth or a different number of teeth. The first clip jaw may include one to six teeth. The second clip jaw may include one to six teeth. The first clip jaw may include one or two teeth and the second clip jaw may include one or two teeth. The first clip jaw and the second clip jaw may each include two teeth. The first clip jaw may include two teeth and the second clip jaw may include one tooth. Different types, shapes, and surfaces of teeth may be effective for attaching to different types of tissue. The teeth of the first clip jaw and the second clip jaw may include a “needle” type pointed tip for piercing the tissue with minimal damage. The teeth of the first clip jaw and the second clip jaw may have needle pointed tips or flat panels with a gripping surface. Alternatively, the teeth of the first clip jaw and the second clip jaw may be flat panels with a gripping surface, such as soft bumps on an interior surface, that grasps tissue without piercing it. This grasping may gently squeeze using flat panels or may be rounded to form a “cage” around the tissue. Alternatively, the teeth may comprise sharp tipped prongs that pierce tissue but do not sever the tissue. Alternatively, the teeth may comprise non-piercing or rounded prongs that gently grab the tissue specimen and do not sever the tissue. The teeth of the first clip jaw and the second clip jaw may include a gripping surface on an inner surface of the clip to increase adherence to a tissue specimen, The tension of spring may be calibrated to ensure adequate pressure to grip tissue with minimal tissue damage.

The tissue orientation clip may include a retaining clip received by the first clip jaw and the second clip jaw. The retaining clip may include a removal tab for removing the retaining clip from the clip jaws prior to use.

The first and second lateral handles may be the same or different shapes. One or both of the lateral handles may be flared, cuboid or V-shaped. The second lateral handle may be V-shaped and define a cutout having a substantially similar dimension as one tooth of the second clip jaw. The first lateral handle may be cuboid or substantially cuboid.

The tissue orientation clip may have a plurality of posts extending therefrom. The posts may be coupled to and extending from the first lateral handle, the second lateral handle, both handles, or from one or both of the first and second clip jaws. The posts may be provided to allow the clips to be quickly and easily grasped by a hand or instrument for removed from the tissue specimen without further tissue damage. To improve this further, the surface of the posts may be textured to facilitate the secure grasp. When the posts extend from one or more of the first and second lateral handles, the posts may provide a surface for operation by fingers to squeeze and move the clip to the open position.

The posts, whether extending from on one or more of the first and second lateral handles or from one or more of the first and second clip jaws, may also be configured to be received by one or more tracks in a cartridge lumen of a delivery device. This allows efficient and secure packing of multiple tissue orientation clips within the cartridge lumen, and also a smooth advancement and expulsion of the clips from the cartridge lumen to the target site.

In certain embodiments, one or both of the first lateral handle and the second lateral handle may include an elongate extension post coupled thereto at the end thereof. The elongate extension post may extend axially and perpendicular to a longitudinal axis of the respective first lateral handle or second lateral handle. Each of the first and second elongate extension posts may have extension post ends at the ends thereof. In another embodiment, the second clip jaw has posts extending therefrom. The second clip jaw may have a first and a second post extending therefrom and in opposite directions, optionally configured to be received by a track in a cartridge.

Another embodiment is a tissue orientation clip delivery system for delivering a single tissue orientation clip comprising a delivery device and a tissue orientation clip. Delivery of the clip may be to a target site, such as a tissue specimen within a patient. The patient may be any animal, a mammal, or a human. The delivery device for a single tissue orientation clip includes: an outer sheath; and a plunger disposed in the outer sheath. The delivery device may also include a clip shield coupled to the outer sheath. The tissue orientation clip is positioned adjacent to a distal end of the plunger, and at least one of the first and second lateral handles of the tissue orientation clip may be coupled within the clip shield. The first lateral handle and the second lateral handle of the tissue orientation clip may be coupled within the clip shield. In an embodiment, the clip shield constrains the clip handle(s) and biases the loaded clip in an open, non-deployed position. The clip shield may include a detent circumferentially disposed on an inner surface of the clip shield for biasing a loaded clip in the open position.

The clip shield may include one or more tracks along the length of the clip shield and parallel to the direction of the length of the plunger. The clip shield may include two tracks, an upper track and a lower track. The track(s) may be configured to receive one or more posts, or a first lateral handle or a second lateral handle of the tissue orientation clip. In an embodiment, the second clip jaw includes at least one post extending therefrom, and wherein the at least one post is received by a track within the clip shield. The second clip jaw may include two posts extending in opposite directions, and each of the two posts is received by a lower track within the clip shield. In an embodiment, one or both of the first and second lateral handles of the tissue orientation clip are received by a track within the clip shield. The first lateral handle may be received by a track within the clip shield, for example an upper track within the clip shield.

The proximal end of the outer sheath may include a finger-graspable handle. A proximal end of the plunger includes a clip release button. The clip shield may be constructed of the same as or different material than the finger-graspable handle, which may be the same as or different from the material of the clip release button. In an embodiment, the clip shield, finger-graspable handle and clip release button are constructed of the same material. Each of the clip shield, finger-graspable handle and clip release button may be constructed of an aromatic polyester (e.g., PET, PETG), polystyrene (PS), polymethylmethacrylate (PMMA), acrylonitrile butadiene styrene (ABS), polycarbonate (PC), or any combination thereof.

The delivery device may further include a spring surrounding an outer circumference of the plunger and disposed between the clip release button and the outer sheath. Any coiled spring to provide the bounce back and proper tension may be used in accordance with the disclosure.

The delivery device may by about 10 inches to about 16 inches in length, or about 12 inches to about 15 inches in length from the end of the clip shield to the clip release button. The clip shield may about 0.75 inches to about 1.5 inches in length, or about 1 inch in length. The plunger may be about 10 inches to about 16 inches in length, or about 12 inches to about 15 inches in length.

The tissue orientation clip delivery device for delivering a single tissue orientation clip may comprise a button lock that prevents unintentional deployment of the clip, optionally in combination with a clip lock and/or button lock removal tab that facilitates easy removal of button lock prior to use.

Another embodiment is a tissue orientation clip delivery system housing multiple tissue orientation clips. The system includes a delivery device and a plurality of tissue orientation clips. The delivery device comprises: a cartridge including a distal end and a proximal end, and a cartridge lumen therewithin; a plunger including a plurality of teeth; and a trigger handle including an extension arm and a catch engageable with a tooth of the plurality of teeth. The plurality of tissue orientation clips are housed within the cartridge lumen at a distal end thereof, each of the plurality of tissue orientation clips in contact with an adjacent tissue orientation clip and the most proximal of the plurality of tissue orientation clips in contact with the plunger.

Each of the plurality of tissue orientation clip may include: a first clip jaw including one or more teeth at a distal end of the first clip jaw, a first lateral handle at a proximal end of the first clip jaw, the first lateral handle defining a first spring arm receiving slot therein; a second clip jaw including one or more teeth at a distal end of the second clip jaw, a second lateral handle at a proximal end of the second clip jaw, the second lateral handle having a second spring arm receiving slot therein; and a spring including a first spring arm received in the first spring arm receiving slot and a second spring arm received within the second spring arm receiving slot. The plurality of tissue orientation clips may be biased in the open position in the cartridge lumen, or biased in the closed position in the cartridge lumen. The distal end of the cartridge may have an inner diameter that is larger than a height of each tissue orientation clip in an open position to permit deployment of each tissue orientation clip.

The cartridge lumen may include one or more tracks along the length of the cartridge. The cartridge lumen may include two tracks along the length of each of the opposing sides of the cartridge. The cartridge lumen may include two tracks, an upper track and a lower track. The track(s) may be configured to receive one or more posts or one or more of the first and second lateral handles of each of the tissue orientation clips.

Each of the plurality of tissue orientation clips may include a post extending therefrom, and an end of each post may be received by a track in the cartridge lumen. In an embodiment, the second clip jaw includes at least one post extending therefrom, and wherein the at least one post is received by a track in the cartridge lumen. The second clip jaw may include two posts extending in opposite directions, and each of the two posts is received by a lower track in the cartridge lumen. In an embodiment, one or both of the first lateral handle and second lateral handle of the tissue orientation clip are received by a track in the cartridge lumen. The first lateral handle may be received by a track within the cartridge lumen, for example an upper track in the cartridge lumen. The second lateral handle may be received by a second track within in the cartridge lumen, for example a lower track in the cartridge lumen. The first lateral handle may be received by an upper track within the cartridge lumen, and a post on the second clip jaw may be received by a lower track in the cartridge lumen.

In an embodiment, the first lateral handle and the second lateral handle of each of the tissue orientation clips includes a first elongate extension post and a second elongate extension post respectively coupled thereto, with each of the first and second elongate extension posts extending axially and perpendicular to a longitudinal axis of the respective first lateral handle or second lateral handle. The cartridge lumen includes upper tracks along the length of each of the opposing sides of the cartridge and lower tracks along the length of each of the opposing sides of the cartridge, and the upper tracks and lower tracks receive the first and second elongate extension posts.

In certain embodiments, in the delivery device, a number of teeth in the plurality of teeth in the plunger corresponds to a number of the plurality of clips in the cartridge lumen. The cartridge may include an aperture for receiving a removable pin configured to prevent plunger from moving prior to use. The plunger may include indicia, which may be numerical, to indicate the number of tissue orientation clips already deployed or remaining in the delivery device. The trigger handle body may include a window configured to display the indicia on the plunger therethrough.

The plunger may include numerical or letter indicia (“plunger indicia”) that corresponds to indicia on the tissue orientation clip that is next to be deployed. In an embodiment, the plunger indicia that is displayed through the window corresponds to indicia on the next clip to be deployed. For example, the plunger indicia displayed in the window may be a letter identifying a margin of a tissue specimen, such as “A” for anterior margin, and the next clip to be deployed, i.e., the clip at the distal end of the cartridge, may also be marked with an “A.” The plunger indicia may also include both a number identifying the number of clips that have been deployed as well as a letter indicia to identify a tissue specimen to be marked. In this embodiment, the window may be configured to display both of the numerical and letter indicia on the plunger.

A proximal end of the plunger may extend past the proximal end of the cartridge and be received within an upper channel of the trigger handle.

The plunger may include a plurality of plungers, for example, a first plunger positioned in the cartridge lumen having a distal end contacting the most proximally positioned tissue orientation clip, and a second plunger received within the upper channel of the plunger extension arm and including a distal end that when assembled to the trigger handle is in contact with the proximal end of the first plunger.

The trigger handle extension post may include a trigger handle protrusion matingly receivable by an aperture in the cartridge. The trigger handle may further comprise a trigger handle body having a trigger lever pivotally coupled thereto, the trigger lever biased in a non-actuated position by a compression spring housed within the trigger handle body. The trigger handle may include a stop lever configured to engage ratcheting slots on the plunger to prevent plunger from retracting proximally after being actuated by a trigger pull.

The cartridge may have the same diameter throughout the entire length thereof or it may have a wider opening (larger diameter) at its distal end. The cartridge may be about 4 inches to about 16 inches, about 5 inches to about 10 inches, or about 5 inches to about 8 inches in length. The distal end of the cartridge may have an inner diameter that is larger than a height of each tissue orientation clip in an open position to permit deployment of each tissue orientation clip. The inner diameter of the distal end cartridge may be about 0.25 inches to about 0.75 inches, or about 0.5 inches. A mid portion of the cartridge may have an inner diameter that is smaller than the distal end of the cartridge and is sized to accommodate the tissue orientation clip in the closed position. A body of the cartridge may have an inner diameter smaller than the distal end of the cartridge and sized to accommodate each tissue orientation clip of the plurality of tissue orientation clips in a closed position. In an embodiment, the body of the cartridge has the same inner diameter as the inner diameter of the distal end of the cartridge, and that inner diameter is about 0.25 inches to about 0.75 inches, or about 0.5 inches.

Another embodiment is a kit comprising a delivery device disclosed herein and one or more tissue orientation clips disclosed herein. The tissue orientation clips may be housed in a cartridge lumen, or if the delivery device is configured for delivery of a single clip, the tissue orientation clip may be housed in a clip shield and coupled to an outer sheath.

Yet another embodiment is a method of delivering one or more of the tissue orientation clips disclosed herein to a target site, such as a tissue specimen in a patient, using a delivery system disclosed herein. In an embodiment, the delivery system includes: a delivery device, comprising: a cartridge including a distal end and a proximal end, and a cartridge lumen therewithin; a plunger including a plurality of teeth; and a trigger handle including an extension arm and a catch engageable with a tooth of the plurality of teeth; and a plurality of any of the tissue orientation clips disclosed herein being housed within the cartridge lumen at a distal end thereof, each of the plurality of tissue orientation clips in contact with an adjacent tissue orientation clip and the most proximal of the plurality of tissue orientation clips in contact with the plunger The method includes: providing the delivery system; inserting the delivery system proximate the target site, e.g., into the patient proximate the tissue specimen; and expelling a first tissue orientation clip of the plurality of tissue orientation clips on the tissue site, and from the distal end of the cartridge by actuating the trigger handle to move the catch and the plunger. Expelling the first clip may include: squeezing a trigger lever pivotably engaged to a trigger body; sliding a post extending from the first clip along a track defined along the cartridge with the plunger; and/or transitioning the first clip from an open position to a closed position grasping the tissue specimen.

The method may further include: expelling a second tissue orientation clip of the plurality of tissue orientation clips on a second location on the tissue site, and from the distal end of the cartridge by actuating the trigger handle to move the catch and advance the plunger. Advancing the plunger may push the first clip out of the cartridge and move a second tissue orientation clip to the distal end of the cartridge. Expelling the second clip may also include: squeezing a trigger lever pivotably engaged to a trigger body; sliding a post extending from the first clip along a track defined along the cartridge with the plunger; and/or transitioning the second clip from an open position to a closed position on the tissue specimen. This step of expelling clips may be repeated one to eight times, or until all of the clips in the delivery device have been expelled from the cartridge. After the desired number of clips have been expelled, the method comprises withdrawing the delivery device from the patient.

The method may further include, prior to expelling the first clip, removing a pin inserted within the cartridge. The method may further include preventing a proximal movement of the plunger through the engagement of a stop lever of the trigger handle and a ratcheting slot of the plunger. The method may further include changing a number displayed in a window defined by the trigger handle contemporaneous with expelling the first clip.

Yet another embodiment is a method of delivering one tissue orientation clip to a target site, such as a tissue specimen in a patient, using a delivery system. The delivery system may comprise: a delivery device that delivers one tissue orientation clip, the delivery device including: an outer sheath, a finger-graspable handle engaged to the outer sheath, a plunger having a proximal end and a distal end, the plunger disposed on the outer sheath, and a clip release button engaged to the proximal end of the plunger; and a tissue orientation clip positioned adjacent a distal end of the delivery device and coupled to the delivery device. Any of the tissue orientation clips disclosed herein may be used in this embodiment. This method may include: providing the delivery system; inserting the delivery system in proximity to the target site; actuating the clip release button on the delivery device to move the plunger in the distal direction; and expelling the tissue orientation clip from a distal end of the plunger.

The delivery device may further include a spring engaged between the finger-graspable handle and the clip release button, the spring providing a biasing force against the clip release button in a proximal direction, and the plunger being received through the sheath, the handle, and the spring. In this embodiment, by actuating the clip release button, the biasing force of the spring is overcome to move the plunger in the distal direction; and expel the tissue orientation clip from the outer sheath through movement of the plunger.

It is to be understood that the disclosure is not limited in its application to the details of construction and the arrangements of components set forth in the following description or illustrated in the following drawings. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof is meant to encompass the items listed thereafter and equivalence thereof as well as additional items. The terms “connected,” “coupled,” and “mounted” and variations thereof are used broadly and encompass direct and indirect connections, couplings, and mountings. In addition, the terms “connected” and “coupled” and variations thereof are not restricted to physical or mechanical connections or couplings. To show how the disclosure may be carried into effect, reference will now be made to the following figures, which are included herein as non-limiting examples of embodiments of the disclosure.

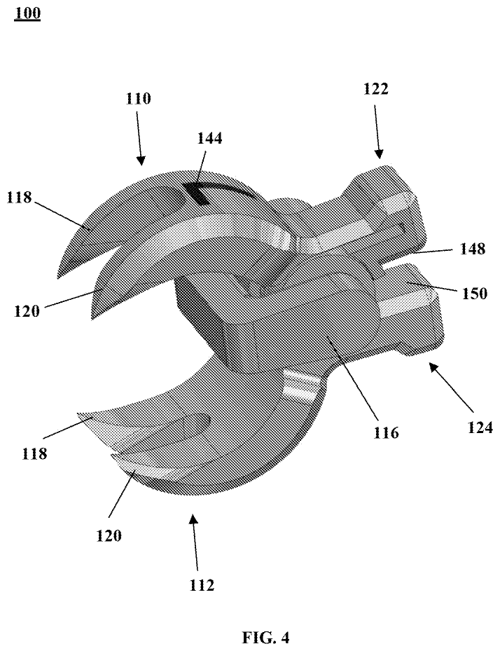

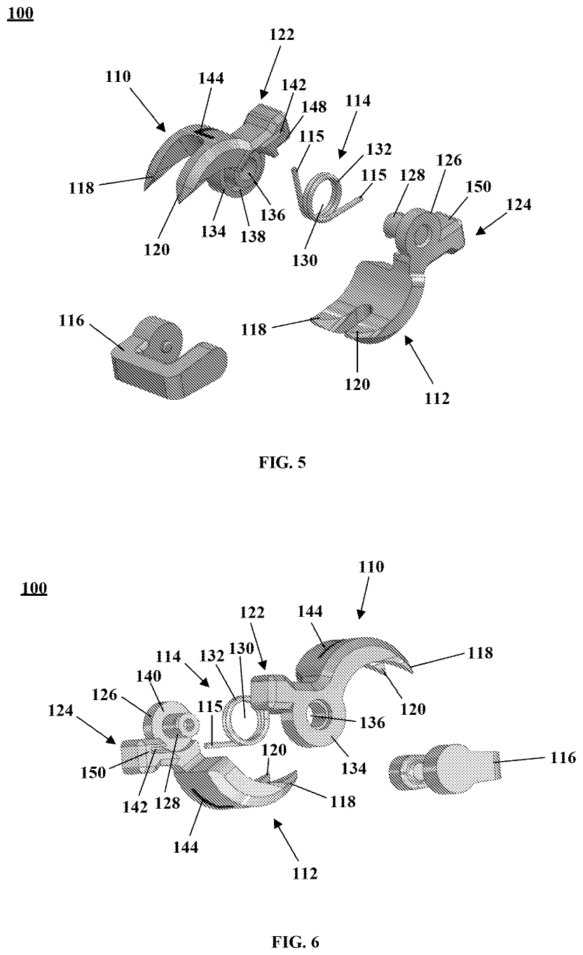

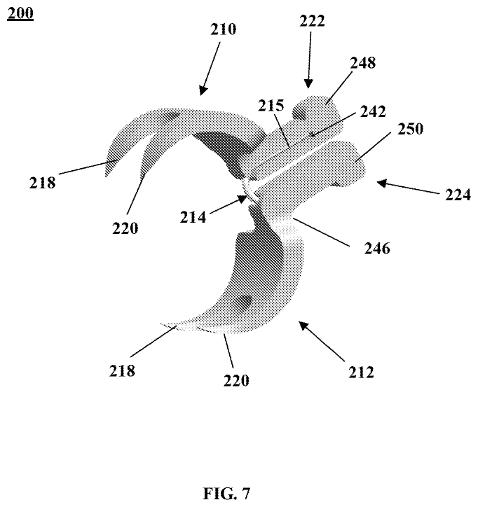

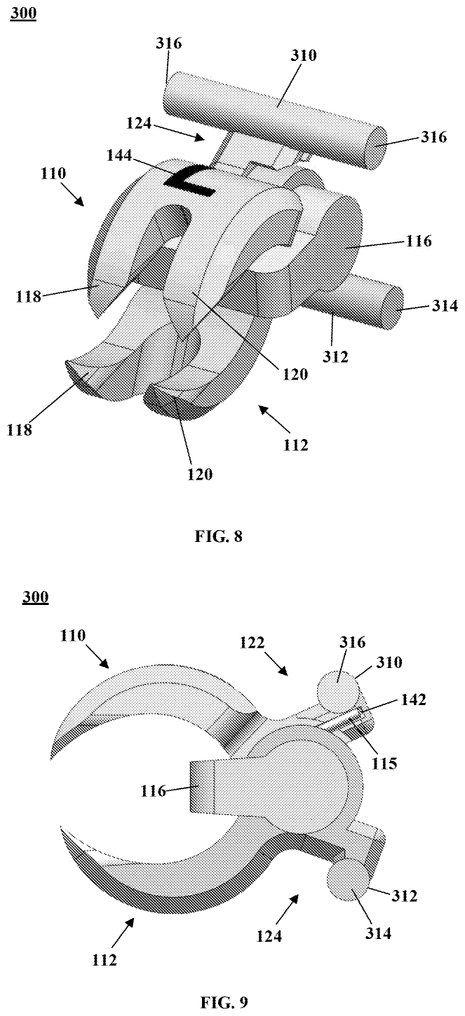

Referring generally now to FIGS. 4-6, one embodiment of a tissue orientation clip 100 in accordance with the disclosure will now be disclosed. FIGS. 5 and 6 are exploded views of one aspect of tissue orientation clip 100. Tissue orientation clip 100 broadly includes first clip jaw 110, second clip jaw 112 and spring 114. Spring 114 may be a torsion spring, as shown, or may be a simple, U-shaped in cross section spring 214 as shown in FIG. 7. Spring 214 includes first and second opposing spring arms 215. Optional retaining clip 116 offers a more secure assembly, preventing clip 100, (or 300 in FIG. 8) from unintended deployment prior to use and may feature removal tab (not shown) extending from retaining clip 116 to securely remove the retaining clip from its retaining position. First clip jaw 110 and second clip jaw 112 each include a first tooth 118 and a second tooth 120 for securely holding the tissue specimen. While two teeth 118, 120 are depicted, those of skill in the art will appreciate that fewer or more than two teeth per clip are contemplated and fall within the disclosure. Teeth 118, 120 are designed to be minimally invasive. First and second teeth 118, 120 may include “needle” type pointed tips for piercing the tissue with minimal damage. First and second jaw arms may include indicia 144 (shown in FIG. 4 as an “L,” by way of example, to represent the lateral margin) to indicate tissue orientation. Indicia 144 may be radiopaque and may indicate an S, I, P, M, A or L. Those of skill in the art will also appreciate that other symbols, including those that do not represent letters or numbers, may be used to indicate the margins or alternatively additional symbols may be used to indicate margins other than the foregoing. Numbers, letters or symbols may also be used to indicate clinical areas of interest on the specimen.

As seen in FIGS. 4-6, first and second jaw arms 110, 112 include first and second lateral handles 122, 124 at a proximal end thereof. First and second lateral handles 122, 124 are easily graspable using gloved fingers or an instrument such as a hemostat after clip 100 is applied to a tissue specimen. Squeezing first and second lateral handles 122, 124 using either fingers or an instrument allows the clips to be quickly and easily removed from the specimen without further tissue damage. Textured surfaces 154, 156 facilitate secure grasp of first and second lateral handles 122, 124 by gloved hands or instrument. Alternatively, those of skill in the art will appreciate that the clip 100 may be removed from the surgical cavity with a hemostat or similar instrument. Second lateral handle 124 includes second circular body 126 having a post 128 projecting therefrom. Second circular body 126 has a diameter slightly larger than a void 130 formed in spring 114. First lateral handle includes first circular body 134 having post receiving hole 136 therein. First circular body 134 includes a recessed well 138 which receives first and second circular bodies 134, 126 of spring 114. When tissue orientation clip 100 is assembled, a first surface of outer circumferential body 132 of spring 114 is received in recessed well 138. Post 128 is received in post receiving hole 136. Void 130 fits over post 128. A second surface of first clip jaw outer circumferential body 134 which houses spring 114 lies adjacent to second clip jaw inner surface 140 of second circular body 126 creating a tight friction fit. Post 128 locks into post receiving hole 136, thereby holding first clip jaw 110 and second clip jaw 112 together around spring 114. Optional retaining clip 116 is received over first and second circular bodies 126, 134 to create a more secure fit.

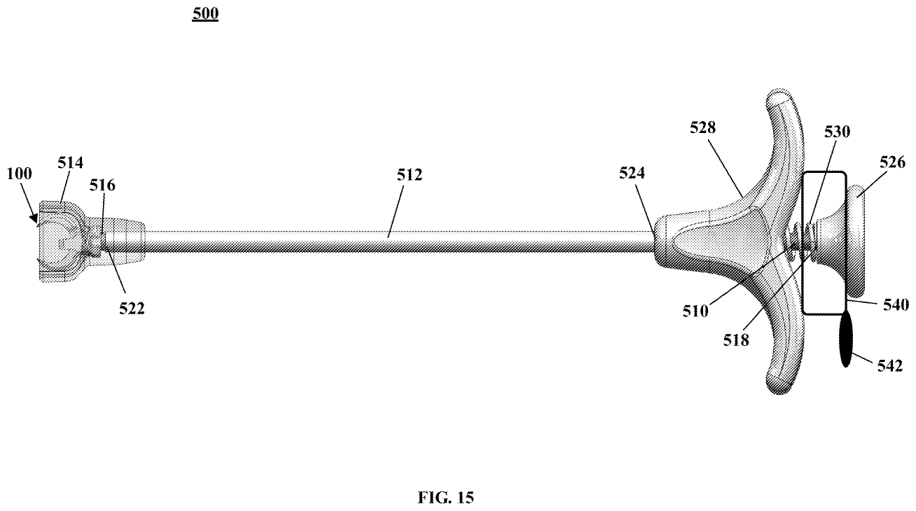

Spring 114 includes a pair of opposing spring arms 115 each of which is received by a slot 142 in lateral handles 122, 124. Spring 114 biases clip 100, when assembled, in the closed position so that when clip 100 is expelled from a single-clip delivery device, such as one shown in FIG. 15-17, the clip closes automatically.

In FIG. 7, a second embodiment of a tissue orientation clip 200 in the open position is shown. Like parts are indicated with like reference numerals. Tissue orientation clip 200 broadly includes first clip jaw 210, second clip jaw 212 and spring 214. Spring 214 is a U-shaped in cross section spring 214. Spring 214 includes a pair of spring arms 215 that are positioned in opposing slots 242 in first and second clip jaw 210, 212 and bias the clip (and teeth) in the closed position (not shown). First clip jaw 210 and second clip jaw 212 each include a first tooth 218 and a second tooth 220 for securely holding the tissue specimen. While two teeth 218, 220 are depicted, those of skill in the art will appreciate that more or less than two teeth per arm are contemplated and fall within the disclosure. One or both of first and second clip jaws 210, 212 may include indicia (not shown) to indicate tissue orientation. First and second clip jaws 210, 212 include first and second lateral handles 222, 224 at a proximal end thereof. First and second lateral handles 222, 224 are designed to be easily grasped using gloved fingers or an instrument after the clip 200 is applied to a specimen. Squeezing the first and second lateral handles 222, 224 allows the clip to be removed from the specimen quickly and easily without damaging the tissue. First and second clip jaws 210, 212 are integrally joined; they may also be joined by optional living hinge 246 to aid in manufacturing and assembly.

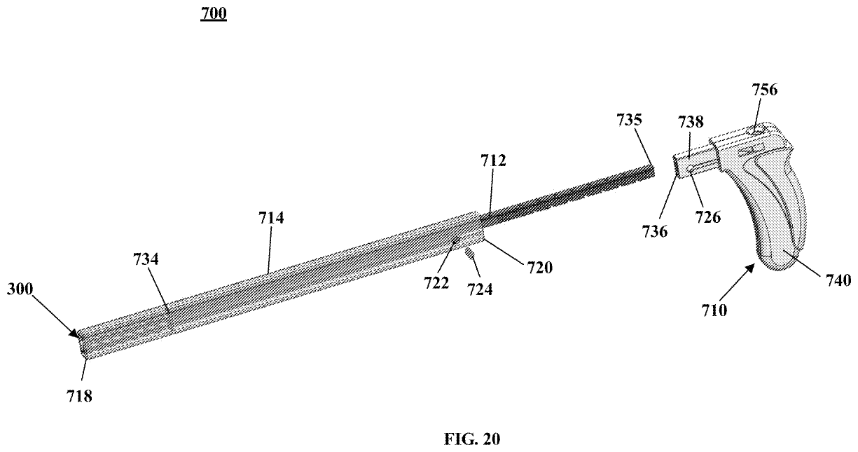

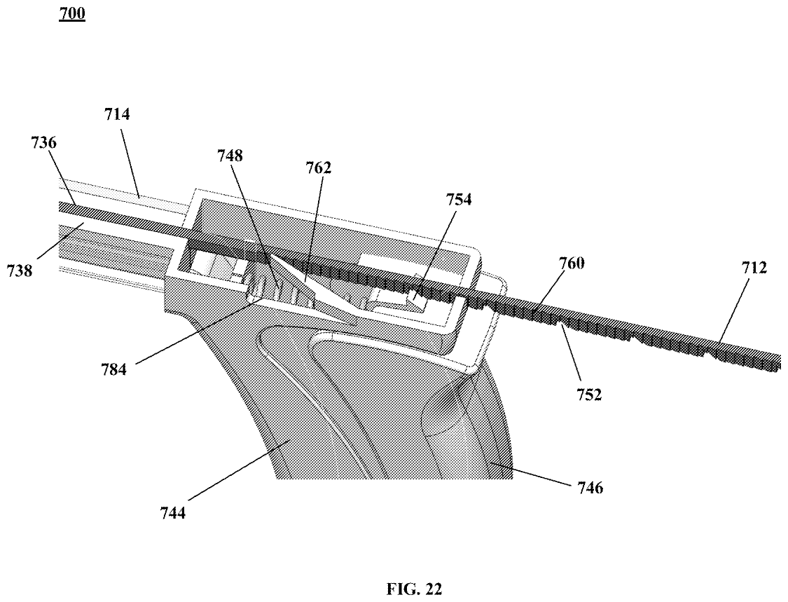

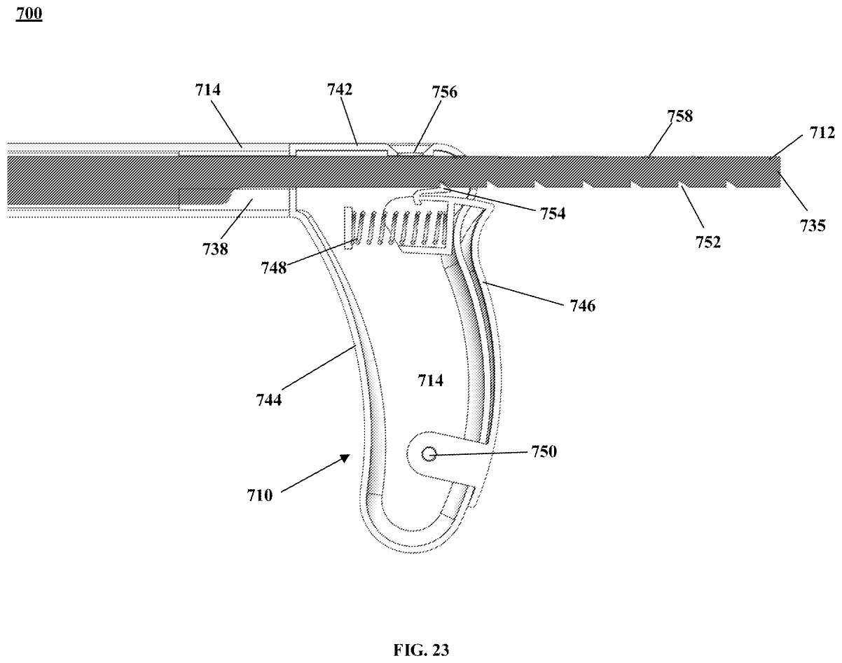

FIGS. 8 and 9 illustrate a third embodiment of a tissue orientation clip 300 designed optionally for use in a delivery device for multiple tissue orientation clips, such as one shown in FIGS. 20-26. Tissue orientation clip 300 is substantially similar to tissue orientation clip 100 and like features have been numbered with like reference numerals. In this third embodiment, first and second clip jaws 110, 112 include first and second lateral handles 122, 124 at a proximal end thereof. First and second lateral handles 122, 124 include first and second extension posts 310, 312 extending therefrom. Each of the extension posts 310, 312 has two opposing extension post ends 314, 316. Extension posts 310, 312 are easily graspable using gloved fingers or an instrument such as a hemostat after clip 300 is applied to the specimen. As first and second extension posts 310, 312 are squeezed together circular body 126 rotates on post 128 so that extension posts come closer together and teeth 118 and 120 become further separated. Extension posts 310, 312 may be operated using either fingers or an instrument, allowing the clips to be quickly and easily removed from the specimen without further tissue damage. The surface of first and second extension posts 310, 312 may be textured to facilitate secure grasp of the first and second extension posts 310, 312 by gloved hands or instrument.

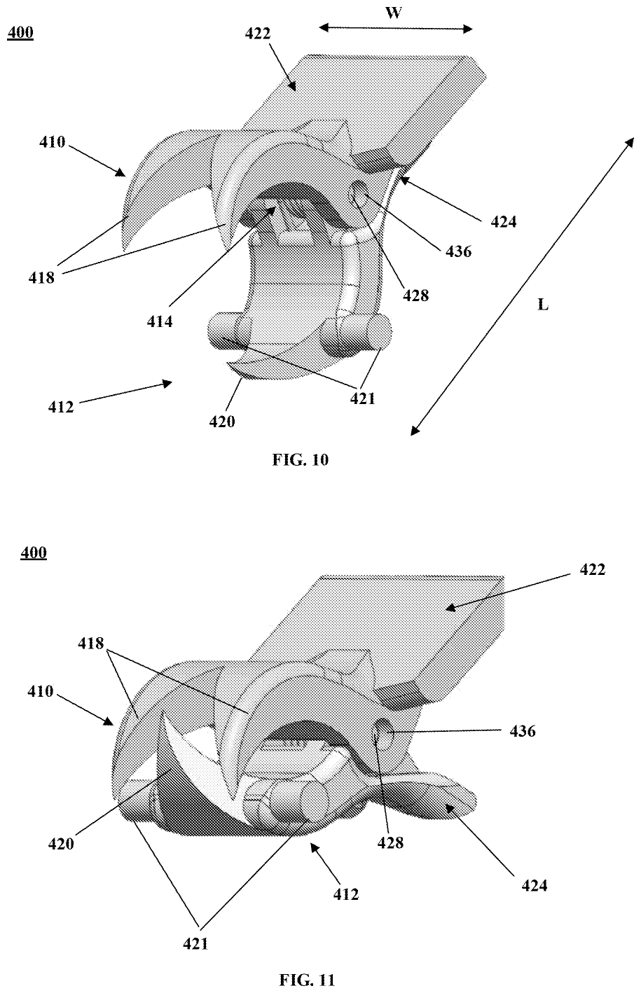

A fourth embodiment of a tissue orientation clip 400 is shown in FIGS. 10-14. First clip jaw 410 has two teeth 418 while second clip jaw 412 has one tooth 420. Teeth 418 of first clip jaw 410 defines a space therebetween that can be sized to receive tooth 420 of second clip jaw 412 when clip 400 is in a closed position as shown in FIG. 11. Second clip jaw 412 includes posts 421 transversely extending from tooth 420. Posts 421 are shown substantially cylindrical in shape, but may be constructed of varying lengths and shapes. First lateral handle 422 is substantially cuboid in shape while second lateral handle 424 is V-shaped such that it includes two substantially pyramidal projections 429 having curved ends. Second lateral handle 424 defines a cutout 425 between projections 429. Cutout 425 can be sized to correspond to tooth 420 of second clip jaw 412 so that, for example, when a plurality of clips 400 are adjacent each other, tooth 420 of an adjacent clip 400 may be received in cutout 425.

The width (W) of the tissue orientation clip in this embodiment is 6 mm-9 mm and the length (L) is 10 mm-16 mm, though it is understood that the width and length may be other sizes as disclosed herein. When W is 9 mm, L may be between about 15 mm and 16 mm, and when W is 6 mm, L may be between about 10 mm and 11 mm. The width of the tissue orientation clip between the two teeth 418 of the first clip jaw 410 (not shown) may be smaller than the width (W) of the first lateral handle. For example, when W is 9 mm, the width between the two teeth 418 of the first clip jaw 410 may be 6 mm, and when W is 6 mm, the width between the two teeth 418 of the first clip jaw 410 may be 4 mm.

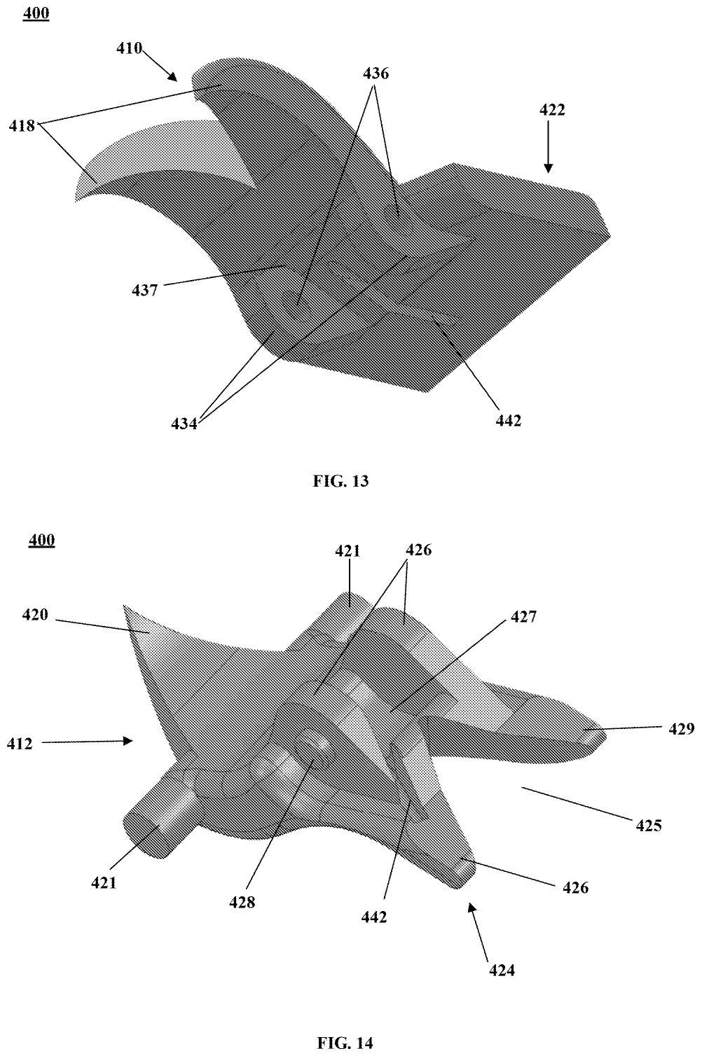

With reference to FIG. 13, first clip jaw 410 includes first body projections 434. Each first body projection 434 includes a protrusion receiving hole 436 therein. Body projections 434 define a first recess 437 therebetween. With reference to FIG. 14, second clip jaw 412 includes second body projections 426. Each second body projection 426 includes a substantially circular protrusion 428 extending therefrom. Second body projections 426 can define a second recess 427 therebetween. Second recess 427 can be sized to receive another component, such as a spring 114.

In an assembled condition, as depicted in FIGS. 10-12, second body projections 426 can be received in first recess 437 such that protrusions 428 are rotatably received in protrusion receiving holes 436. The engagement between protrusions 428 and protrusion receiving holes 436 can allow for relative rotatable movement between first clip jaw 410 and second clip jaw 412 while limiting relative axial movement between first clip jaw 410 and second clip jaw 412.

It is understood that posts 421, first lateral handle 422, second lateral handle 424, projections 426, and other features of the embodiment, may have other shapes, such as being cuboid, pyramidal, or any other prism shape.

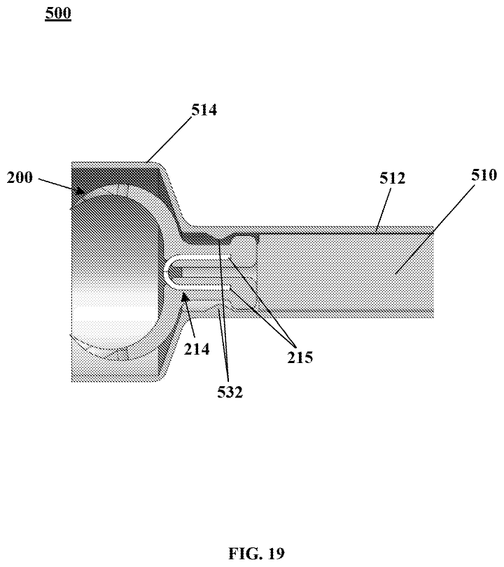

A delivery device for a tissue orientation clip (also referred to herein as a “single-clip delivery device”) 500 in accordance with the disclosure is shown in FIGS. 15-17 and 19. Single-clip delivery device 500 includes plunger 510, outer sheath 512 and clip shield 514. Plunger 510 includes distal plunger end 516 and proximal plunger end 518. Plunger proximal end includes a clip release button 526 that releases clip 100, 200 from the single-clip delivery device 500 by pushing it out of the outer sheath 512. Plunger 510 has an outer diameter that is smaller than the inner diameter of outer sheath 512 so that it fits snugly in the outer sheath 512 lumen 520 but can slide smoothly through the outer sheath 512 lumen 520. Outer sheath 512 includes outer sheath distal end 522 and outer sheath proximal end 524. Outer sheath proximal end 524 includes a handle 528 that is graspable by a user between the index finger and the middle finger while the user's thumb controls the clip release button 526. As depicted, handle 528 is T-shaped but those of skill in the art will appreciate that the handle could also include other shapes such as circular so long as it is graspable between fingers such as the index finger and middle finger. Functionally, the two fingers push in the direction from distal to proximal while a third finger, such as the thumb, pushes in the direction from proximal toward distal. The length of the outer sheath 512 is slightly less than the length of the plunger 510 to accommodate optional spring 530. The length of the outer sheath may be about 0.25 inches to about 2 inches shorter than the length of the plunger. Optional spring 530 may be positioned over the plunger 510 at the proximal end 518 and interposed between the clip release button 526 and handle 528 such that when a user depresses the clip release button 526 to release clip 100, the clip release button automatically retracts proximally to its initial position. Optional spring 530 may be a compression spring or other type of spring known to those of skill in the art. Spring 214 including first and second opposing spring arms 215 is depicted in FIG. 19. Optional button lock 540 prevents clip release button 526 from being deployed prior to use. Button lock removal tab 542 facilitates easy removal of button lock 540 prior to use.

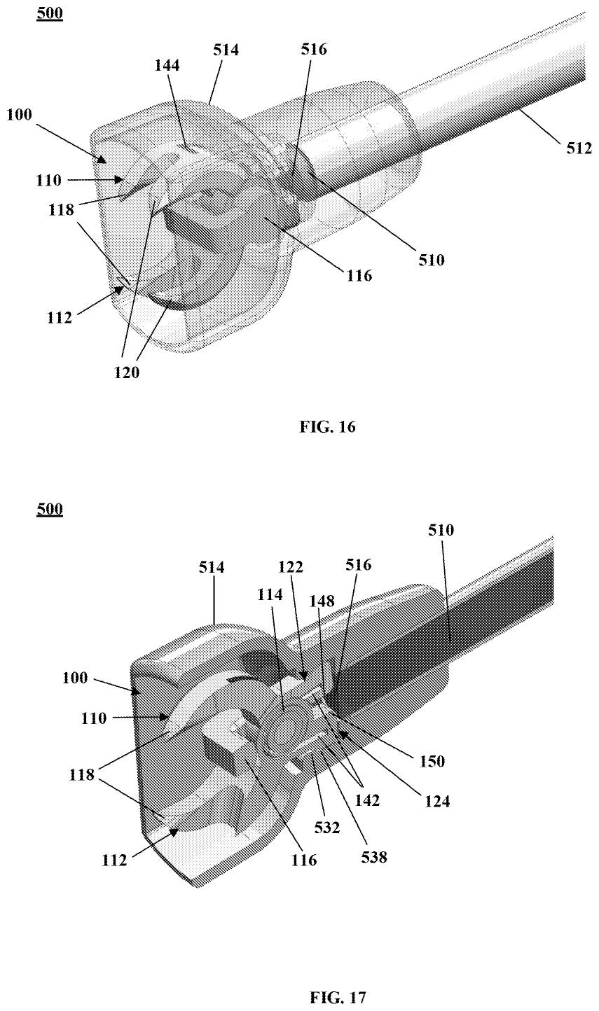

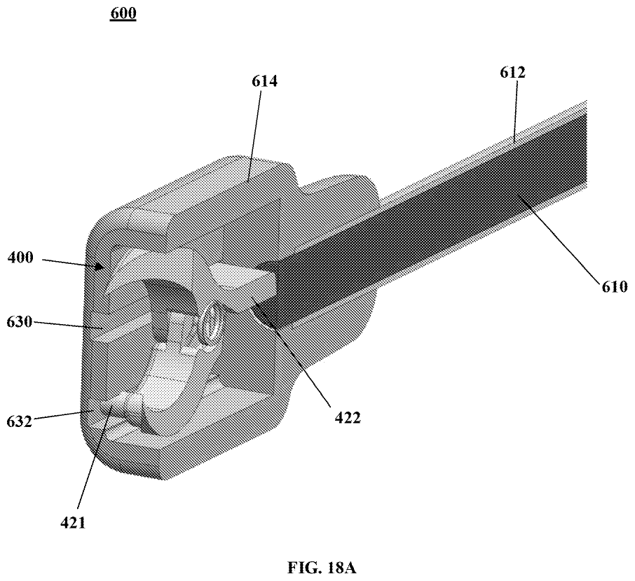

Clip shield 514 is positioned at the distal outer sheath end 522 by means such as chemical bonding, integrally molded, adhesives and the like. As best seen in FIG. 15, the clip shield 514 inner circumferential surface 538 is slightly larger than the width of clip handles 122, 124 at their widest point when the spring is fully compressed, as shown in FIGS. 16 and 17. Clip shield 514 may be constructed of rigid or semi-rigid materials known to those of skill in the art, which oppose the force of spring 114 when clip handles 122, 124 are positioned within first and second interior surfaces 148, 150 adjacent to one another; in other words the “open” position. Protruding detent 532 prevents clip 100, 200 from being accidentally expelled from the clip shield 514 when housed therewithin.

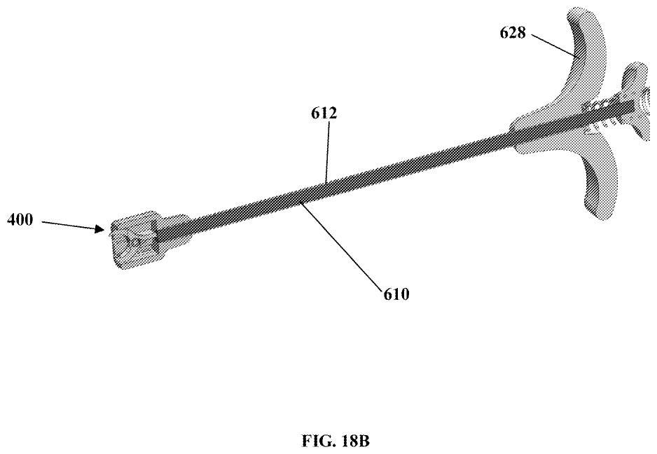

In FIGS. 18A-18B, single-clip delivery device 600 includes plunger 610, outer sheath 612 and clip shield 614. Clip shield 614 includes an upper track 630 and a lower track 632. In these figures, the single-clip delivery device 600 houses tissue orientation clip 400. The upper track 630 receives the first lateral handle 422 of clip 400 and the lower track 632 receives the post 421. Handle 528 is depicted as T-shaped at the opposite end of the plunger from the clip shield 614 and clip 400.

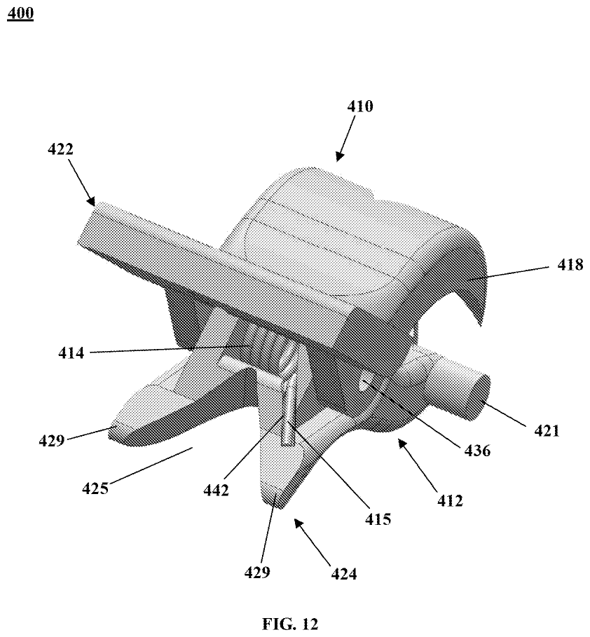

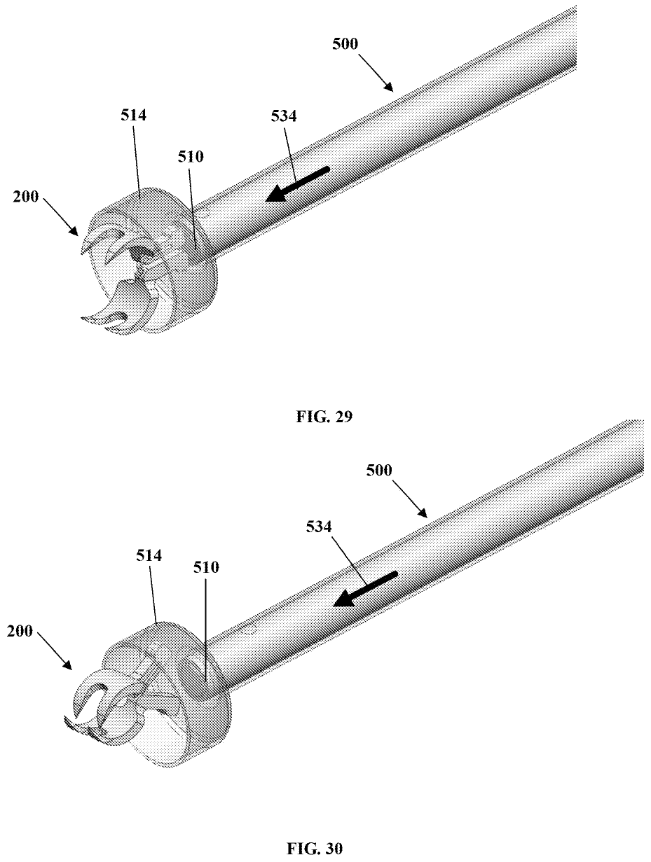

In FIGS. 29-30, the force 534 of the plunger 510 against the clip 200 causes the clip to overcome the restriction of clip shield proximal inner surface 538 and restraint of detent 532 and to exit the clip shield 514 shown in FIGS. 12-17 and 19. Upon exit, spring 114, 214 engages clip jaw 110 and 112 or 210 and 212 into the “closed” position. This action allows the clip to automatically grasp and mark the tissue specimen.

The clips 100, 200, 300, 400 may be sized to be (e.g., to have a width that is) smaller than the inner diameter of the outer sheath 512, 612 herein described and smaller than the inner diameter of the trocar through which the tissue specimen with attached clip will be extracted from the patient.

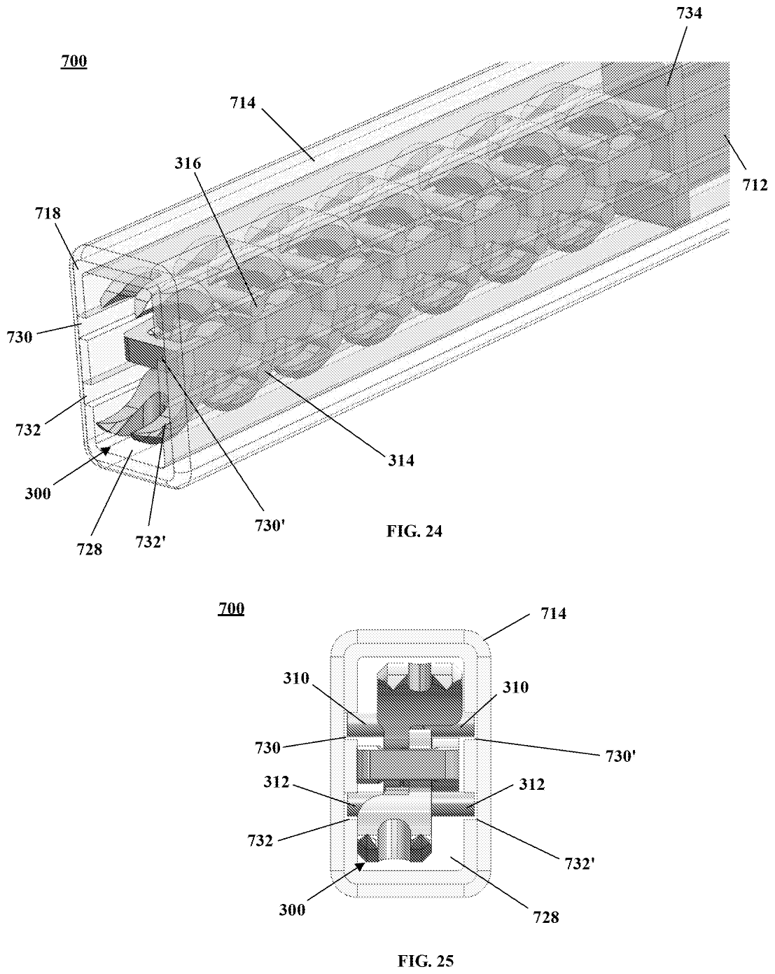

Referring now to FIGS. 20-25, a delivery device for multiple tissue orientation clips (also referred to herein as a “multi-clip delivery device”) 700 is disclosed. Referring to FIG. 20, the multi-clip delivery device 700 broadly includes trigger handle 710, plunger 712 and cartridge 714. Cartridge 714 is configured to house and is depicted housing multiple clips 300 at a distal end 718 thereof. The proximal end 720 of cartridge 714 includes an aperture 722 for receiving removable pin 724. Removable pin 724 prevents plunger 712 from unintentional movement, such as, while in transit. Trigger handle 710 includes a trigger handle protrusion 726 that mates with aperture 722 when assembled and operational. As shown in FIG. 24, cartridge 714 includes a cartridge lumen 728 therewithin which receives plunger 712 and multiple clips 300 positioned in the distal end 718 of cartridge 714. The distal end 734 of plunger 712 abuts the most proximal clip 300 positioned in lumen 728.

Referring also to FIGS. 20-25, the trigger handle 710 and operation of the multi-clip delivery device will be disclosed. The distal end 734 of plunger 712 abuts the most proximal tissue orientation clip 300 in cartridge 714. The proximal end 735 of plunger 712 includes a plurality of teeth 752, the number of which correspond to the number of clips 300 loaded in the delivery device 700 and as indicated in the top surface of plunger 712. Plunger 712 also includes a number of ratcheting slots 760 that mate with integrally molded stop lever 762. When a user squeezes the lever 746 on trigger handle 710 the plunger 712 advances through channel 736, by about 10 mm to about 15 mm, or about 13 mm, with each trigger pull while stop lever 762 traverses a corresponding number of ratcheting slots 760. To further track the delivery of clips 300, stop lever 762 may be seen through stop lever window 784. Trigger handle 710 includes a trigger handle protrusion 726 on extension arm 738 that mates with aperture 722 when assembled and ready for operation. Trigger handle 710 broadly includes main handle body 740 having an upper body 742 and lower body 744 portions and extension arm 738. Cartridge 714 includes a lumen 728 therewithin which receives plunger 712 and multiple clips 300 positioned in the distal end 718. The proximal end 735 of plunger 712 is received through a channel 736 in extension arm 738 that extends from the upper body 742 of trigger handle 710. Trigger handle 710 includes lever 746 that is biased in the non-actuated position by handle spring 748. Lever 746 is pivotally coupled to main handle body 740 by pivot pin 750. Lever 746 includes an integrally-molded catch 754 in the upper body 742 that mates with a single tooth of the plurality of teeth 752 per trigger pull actuation.

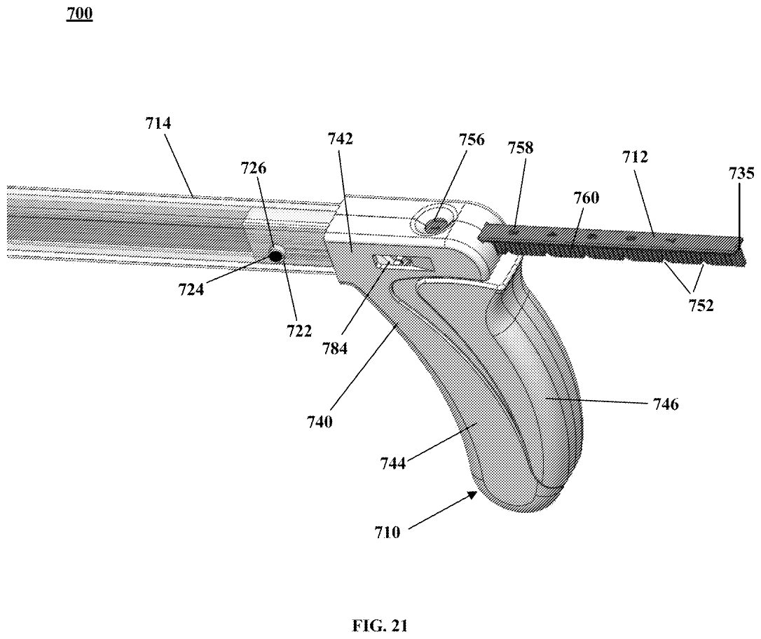

As seen in FIG. 21, trigger handle 710 includes a window 756 in the upper trigger handle body 742 which enables a user to view indicia on the plunger, for example, the number of clips initially loaded in the cartridge 714 and how many clips remain as the clips are pushed through the cartridge 714 during operation. The number of clips 758 are shown in chronological order. In other words, in FIG. 24, seven clips are loaded in the device and have not yet been deployed. As the trigger is pulled and the first clip is expelled from the device and deployed by the user, the number changes in the window 756, for example, from 1 to 2. However, those of skill in the art will appreciate that the numbers may be reversed with the first number being the initial number of clips loaded in the device (i.e., seven) with the number remaining as the plunger is advanced through cartridge 714. It is understood that the indicia on the plunger may be numerical as discussed above, or may be a letter identifying a margin of a tissue specimen, or a symbol representing a margin or a clinical area of interest.

In operation, a user positions cartridge 714 proximate a tissue specimen to be marked and squeezes the lever 746 on trigger handle 710, which advances plunger 712 through channel 736 by about 13 mm with each trigger pull. As plunger 712 advances through channel 736 it pushes a clip 300 out of cartridge 714. When clip 300 exits cartridge 714 the tracks 730, 732 are no longer biasing the clip 300 in the open position and clip 300 automatically enters a closed position, for example, grasping onto a tissue specimen, upon exiting cartridge 714 due to spring 114 biasing clip jaws 110, 112 together. When a clip 300 exits the cartridge 714 and the next clip advances, the engagement between ratcheting slots 760 and stop lever 762 prevents plunger 712 from reversing when the trigger return to the loaded position.

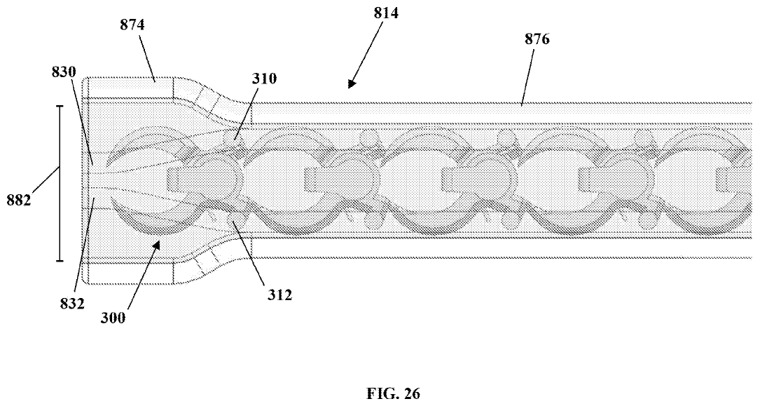

Referring to the multi-clip delivery device 700 in FIGS. 24-25, cartridge lumen 728 includes upper and lower tracks 730, 730′, 732, 732′ along the length of the cartridge lumen. Upper and lower tracks 730, 730′, 732, 732′ slidingly receive opposing ends 314, 316 of posts 310, 312 from FIGS. 8-9. A plurality of clips 300 are loaded in the open position while tracks 730, 730′, 732, 732′ maintain clips 300 in the open position while in the cartridge 714. Seven clips are shown in the cartridge lumen in FIG. 24 though it is understood that more or fewer clips may be loaded into the cartridge. For example 5 to 20 clips may be loaded into the multi-clip delivery device. Tissue orientation clip 300 is sized to be slightly smaller than the inner diameter of cartridge lumen 728 and the inner diameter of the trocar through which the tissue specimen with attached clip will be extracted from the patient.

Another embodiment of a multi-clip delivery device is shown in FIG. 26 with modified cartridge 814, for use instead of, e.g., 714, with delivery device 700. Clips 300 are identical to clips used with multi-clip delivery device 700 but are loaded into cartridge 814 in the closed position. Upper and lower tracks 830, 832 receive posts 310, 312 and maintain the clips 300 in the closed position in cartridge 814. The distal portion 874 of cartridge 814 flares into an open configuration to accommodate the outer diameter of clips 300 as they open and exit the cartridge 814 as further explained below. The flared configuration of distal portion 874 of cartridge 814 has an outer diameter greater than the outer diameter of main cartridge body 876. The inner diameter 882 of flared distal end 874 is greater than the outer diameter of first and second clip jaws 110, 112 of clips 300 in the open position. As shown in FIG. 26, upper and lower tracks 830, 832 converge as they proximate the distal portion 874 of cartridge 814. This convergence forces posts 310, 312 to squeeze together which forces the opening of first and second clip jaw 110, 112 of each clip 300 as clip 300 exits the distal end 874 of cartridge 814. As the clip 300 exits the cartridge 814 and contacts the tissue specimen, first and second clip jaws 110, 112 close as posts 310, 312 revert to the original position.

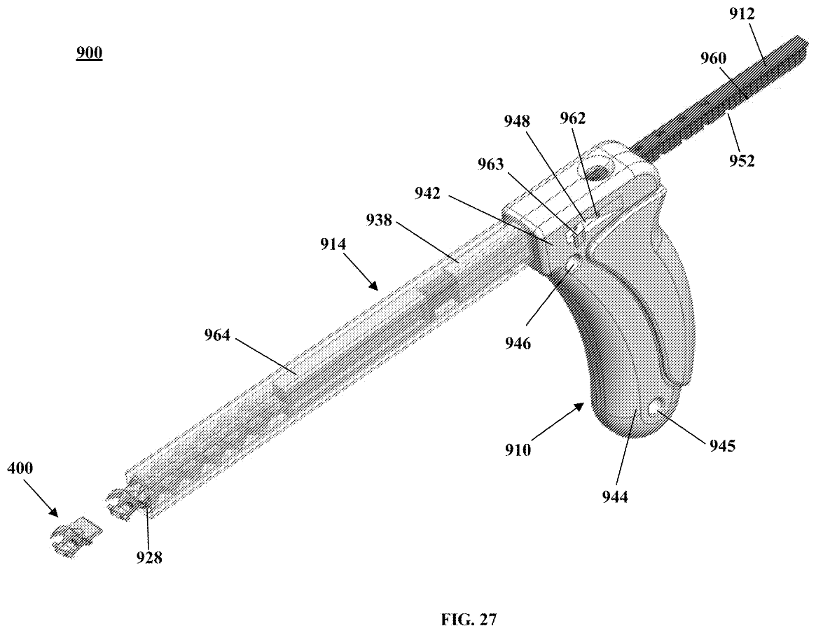

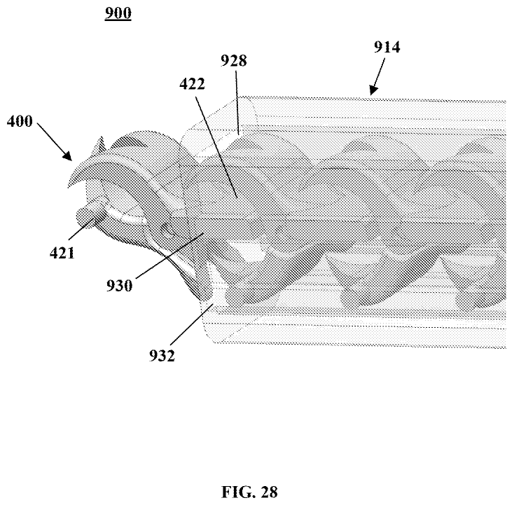

FIG. 27 depicts another embodiment of a multi-clip delivery device 900. Multi-clip delivery device 900 is substantially similar to multi-clip delivery device 700, with like features having like reference numerals except under the “900” series of numbers rather than “700” series, with differences hereinafter disclosed. One difference is the incorporation of a shortened first plunger 912 having teeth 952 and ratcheting slots 960. For example, the proximal end of the shortened first plunger may extend through the channel in upper body 942 of trigger handle 910 while the distal end of the shortened first plunger extends through extension arm 938 in a snap-fit arrangement similar to multi-clip delivery device 700. Second plunger 964 abuttingly mates against the distal end of first plunger 912 so that as the trigger handle 940 is pulled or actuated, the first plunger 912 moves distally and in turn causes second plunger 964 to move distally and expel each tissue orientation clip 400 as previously disclosed.

Stop lever 962 includes an extension 963 extending from stop lever 962 such that extension 963 extends out of stop lever window 948. Extension 963 is substantially L-shaped in a proximal direction. Extension 963 can be shaped to be engaged by a user or a tool so that, for example, extension 963 can be pulled in a proximal direction to disengage stop lever 962 from ratcheting slots 960 and allow plunger 912 to be pulled in a proximal direction. Extension 963 permits resetting of the plunger upon reloading.

Trigger handle 910 defines holes 945, 946. Holes 945, 946 can allow for a fastener (such as a screw, nail, bolt, or the like) to be received therein to maintain the assembly of trigger handle 910.

In the multi-clip delivery device 900, cartridge lumen 928 includes upper and lower tracks 930, 932 on opposing sides of the cartridge lumen along the length of the cartridge lumen. Upper track 930 (and 930′ not shown) slidingly receives the first lateral handle, as shown, for example in FIG. 10. Lower track 932 (and 932′ not shown) slidingly receives post 421 protruding from the second clip jaw as shown, for example in FIG. 10. A plurality of clips 400 are loaded in the open position while tracks 930, 930′, 932, 932′ maintain clips 400 in the open position while in the cartridge 914. Eight clips are shown in FIG. 27 though it is understood that more or fewer clips may be loaded into the cartridge. For example 5 to 20 clips may be loaded into the multi-clip delivery device. Tissue orientation clip 400 is sized to be slightly smaller than the inner diameter of cartridge lumen 928 and the inner diameter of the trocar through which the tissue specimen with attached clip will be extracted from the patient.

It should be understood that cartridges 714, 814, 914 of delivery devices 700, 900 may include any number of tracks to slidingly receive any number of posts, lateral handles or other features extending from the tissue orientation clips housed within the cartridge. For example, a cartridge can have three sets of tracks along each side of the lumen of the cartridge where the clips received in the cartridge include three posts extending from each side of the clips. Additionally, it is understood that, in other examples, there can be more or less than two holes 945, 946, such as none, one, three, or more.

Although the invention has been described in detail with reference to certain preferred embodiments, variations and modifications exist within the scope and spirit of the invention as described and defined in the following claims.

1. A tissue orientation clip comprising:
a first clip jaw including one or more teeth at a distal end of the first clip jaw, a first lateral handle at a proximal end of the first clip jaw, the first lateral handle defining a first spring arm receiving slot therein;
a second clip jaw including one or more teeth at a distal end of the second clip jaw, a second lateral handle at a proximal end of the second clip jaw, the second lateral handle defining a second spring arm receiving slot therein; and
a spring including a first spring arm received in the first spring arm receiving slot and a second spring arm received within the second spring arm receiving slot.

2. The tissue orientation clip of claim 1, wherein the spring biases the tissue orientation clip in a closed position.

3. (canceled)

4. The tissue orientation clip of claim 3, wherein a protrusion of the second clip jaw is rotatably received in a protrusion receiving hole in the first clip jaw.

5. (canceled)

6. The tissue orientation clip of claim 1, further comprising indicia indicating an orientation of a tissue specimen.

7-10. (canceled)

11. The tissue orientation clip of claim 10, wherein the clip is less than about 3.5 cm in length and less than about 3.5 cm in width.

12. The tissue orientation clip of claim 1, wherein the first clip jaw or the second clip jaw includes two or more posts extending perpendicularly from said first clip jaw or said second clip jaw.

13. (canceled)

14. The tissue orientation clip of claim 1, wherein the first lateral handle and the second lateral handle include a first elongate extension post and a second elongate extension post respectively coupled thereto,
wherein each of the first and second elongate extension posts extends axially and perpendicular to a longitudinal axis of the respective first lateral handle or second lateral handle, and
wherein each of the first and second elongate extension posts has a first extension post end and a second extension post end.

15. (canceled)

16. (canceled)

17. The tissue orientation clip of claim 1, wherein the first clip jaw includes two teeth and the second clip jaw includes one or two teeth.

18. The tissue orientation clip of claim 1, wherein the first lateral handle is substantially cuboid.

19. The tissue orientation clip of claim 1, wherein the clip is made of a material selected from a gamma stable plastic, titanium, or stainless steel.

20. (canceled)

21. A tissue orientation clip delivery system for delivering a tissue orientation clip of claim 1, comprising:
a delivery device comprising:
an outer sheath;
a plunger disposed in the outer sheath; and
a clip shield coupled to a distal end of the outer sheath; and
the tissue orientation clip positioned adjacent a distal end of the plunger, and coupled within the clip shield.

22. (canceled)

23. The system of claim 21, wherein a proximal end of the plunger includes a clip release button.

24. The system of claim 23, further comprising a spring surrounding an outer circumference of the plunger and disposed between the clip release button and the outer sheath.

25. (canceled)

26. The system of claim 21, wherein the first and second lateral handles of the tissue orientation clip are coupled within the clip shield.

27. (canceled)

28. A tissue orientation clip delivery system housing multiple tissue orientation clips of claim 1, comprising
a delivery device comprising:
a cartridge including a distal end and a proximal end, and a cartridge lumen therewithin;
a plunger including a plurality of teeth; and
a trigger handle including an extension arm and a catch engageable with a tooth of the plurality of teeth; and
a plurality of tissue orientation clips housed within the cartridge lumen at a distal end thereof, each of the plurality of tissue orientation clips in contact with an adjacent tissue orientation clip and the most proximal of the plurality of tissue orientation clips in contact with the plunger.

29. (canceled)

30. (canceled)

31. The tissue orientation clip delivery system of claim 28, wherein the trigger handle extension post includes a trigger handle protrusion matingly receivable by an aperture in the cartridge.

32. The tissue orientation clip delivery system of claim 28, wherein the cartridge lumen includes a track along the length of the cartridge.

33-35. (canceled)

36. The tissue orientation clip delivery system of claim 32, wherein each of the plurality of tissue orientation clips includes a post extending therefrom, wherein an end of each post is received by the track.

37-40. (canceled)

41. The tissue orientation clip delivery system of claim 28, wherein the trigger handle further comprises a trigger handle body having a trigger lever pivotally coupled thereto, the trigger lever biased in a non-actuated position by a compression spring housed within the trigger handle body.

42. The tissue orientation clip delivery system of claim 41, wherein the trigger handle includes a stop lever configured to engage ratcheting slots on the plunger to prevent the plunger from retracting proximally after being actuated by a trigger pull.

43. (canceled)

44. (canceled)

45. The tissue orientation clip delivery system of claim 28, wherein a proximal end of the plunger extends past the proximal end of the cartridge and is received within an upper channel of the trigger handle.

46. (canceled)

47. A method of delivering one or more tissue orientation clips of claim 1 to a tissue specimen in a patient, the method comprising:
providing a delivery system that delivers the one or more tissue orientation clips, the system comprising:
a delivery device, comprising:
a cartridge including a distal end and a proximal end, and a cartridge lumen therewithin;
a plunger including a plurality of teeth;
a trigger handle including an extension arm and a catch engageable with a tooth of the plurality of teeth; and
a plurality of the tissue orientation clips housed within the cartridge lumen at a distal end thereof, each of the plurality of tissue orientation clips in contact with an adjacent tissue orientation clip and the most proximal of the plurality of tissue orientation clips in contact with the plunger;
inserting the delivery system into the patient proximate the tissue specimen; and
expelling a first tissue orientation clip of the plurality of tissue orientation clips from the distal end of the cartridge by actuating the trigger handle to move the catch and advance the plunger.

48. The method of claim 47, wherein expelling the first clip includes squeezing a trigger lever on the trigger handle.

49. (canceled)

50. (canceled)

51. The method of claim 47, wherein expelling the first clip includes transitioning the first clip from an open position to a closed position on the tissue site.

52-54. (canceled)

55. The method of claim 47, wherein when the first tissue orientation clip is expelled, a second tissue orientation clip advances to the distal end of the cartridge.
